(19) United States
(12) Patent Application Publication
(10) Pub. No.: US 2011/0319744 A1
(43) Pub. Date: Dec. 29, 2011

(54) PHOTOACOUSTIC IMAGING APPARATUS AND PHOTOACOUSTIC IMAGING METHOD

(75) Inventors: Kazuhiro Tsujita, Kanagawa-ken (JP); Miya Ishihara, Tokorozawa-shi (JP)

(73) Assignee: FUJIFILM CORPORATION, Tokyo (JP)

(21) Appl. No.: 13/167,647

(22) Filed: Jun. 23, 2011

(30) Foreign Application Priority Data
Jun. 24, 2010 (JP) 143374/2010

(51) Int. Cl. A61B 6/00

(52) U.S. Cl. 600/407

(57) ABSTRACT

A region selecting means sequentially selects a plurality of partial regions, into which a range to be imaged of a subject is divided. A light irradiation detecting section detects light which is irradiated onto the subject from a laser light source. A signal obtaining section samples acoustic signals detected by probe elements corresponding to the selected partial region, and stores the acoustic signals in an element data memory. An image constructing section constructs a tomographic image of the subject based on the data read out from the element data memory. A synchronization correction processing section obtains the differences in the timings at which the light irradiation detecting section has detected irradiation of light, and corrects the temporal axes of the sampled data in the element data memory based on the obtained timing differences.

### BACKGROUND OF THE INVENTION

1. Field of the Invention

The present invention is related to a photoacoustic imaging apparatus and a photoacoustic imaging method. More specifically, the present invention is related to a photoacoustic imaging apparatus and a photoacoustic imaging method that performs imaging based on acoustic signals generated by light which is irradiated onto a subject.

2. Description of the Related Art

Photoacoustic imaging, which images the interiors of living organisms utilizing the photoacoustic effect, is known. Generally, in photoacoustic imaging, pulsed laser beams such as laser pulses are irradiated into the living organisms. Biological tissue that absorbs the energy of the pulsed laser beams generate acoustic waves (acoustic signals) by volume expansion thereof due to heat. The acoustic waves are detected by an ultrasound probe or the like, and the detected signals are utilized to enable visualization of the living organisms based on acoustic waves.

Japanese Unexamined Patent Publication No. 2005-21380, for example, discloses an apparatus that generates photoacoustic images. This apparatus performs imaging using a method similar to the line by line method utilized by ultrasound examination apparatuses. That is, light from alight source is guided to biological tissue by a waveguide section, and a pulsed laser light beam is irradiated onto the biological tissue. After irradiation of the pulsed laser beam, acoustic waves are detected by adjacent probe elements corresponding to a predetermined number of channels of an ultrasound probe. The detected acoustic waves are phase matched and added, to enable specification of the depth positions within the organism at which the acoustic waves are generated. The irradiation of the pulsed laser beam and the detection of the acoustic waves are repeatedly executed while shifting the probe elements by single channels (single lines), to construct a photoacoustic image.

In addition to the method described above, there is a known technique that stores data for all probe elements of an ultrasound probe in an element data memory, and performs imaging utilizing the data stored in the element data memory. This type of image construction is disclosed in X. Wang, J. Cannata, D. DeBusschere, C. Hu, J. B. Fowlkes, and P. Carson, “A High Speed Photoacoustic Tomography System based on a Commercial Ultrasound and a Custom Transducer Array”, Proc. SPIE, Vol. 7564, No. 24, Feb. 23, 2010, for example.

Various research reports have been submitted regarding photoacoustic imaging. A problem to be solved to realize practical use of photoacoustic imaging is to increase the speed of imaging. In the apparatus disclosed in Japanese Unexamined Patent Publication No. 2005-21380, only signals corresponding to a single line can be detected with a single light irradiating operation. Therefore, the amount of time required to perform imaging becomes longer as the number of elements (number of channels) of the ultrasound probe increases. It is necessary to shorten the intervals among light irradiating operations in order to increase imaging speed in this apparatus. However, it is necessary to suppress the energy level which is irradiated onto living organisms beneath a certain level, for reasons of safety. For example, there are safety standards with respect to the amount of energy for a single pulse of a pulsed laser beam, and for the number of repetitive pulsed laser beam irradiating operations. Therefore, the intervals among light irradiating operations cannot be shortened beyond a certain degree. Accordingly, there are limits to improvements in the imaging speed of the apparatus disclosed in Japanese Unexamined Patent Publication No. 2005-21380.

Meanwhile, in the technique disclosed by Wang et al., all of the data from the probe elements are temporarily stored in the element data memory, then imaging is performed. In the case that an ultrasound probe has probe elements corresponding to 128 channels, for example, increased imaging speed becomes possible by obtaining data for all 128 channels in parallel. However, an A/D (Analog/Digital) converter will be necessary for all of the channels in the case that this is actually performed. A/D converters that operate in parallel and at high speeds are expensive, and the cost of the system will increase because it will be necessary to provide a great number of such A/D converters.

Here, if data for 64 channels are obtained by a single laser pulse irradiation, and data for 128 channels are obtained by performing such data obtainment twice, the number of A/D converters that operate in parallel can be 64. In this case, the number of A/D converters necessary to obtain data for 128 channels is halved compared to a case in which data for 128 channels are obtained at once, and cost can be reduced. However, if laser pulses are irradiated a plurality of times, there is a possibility that irradiation timings of the laser pulses will be shifted between a first irradiating operation and a second irradiating operation. That is, there is a possibility that jitters will occur in the laser pulses. If jitters occur, errors will be generated between data obtained during the first light irradiating operation and data obtained during the second light irradiating operation, resulting in the image quality of images generated using the data deteriorating.

### SUMMARY OF THE INVENTION

The present invention has been developed in view of the foregoing circumstances. It is an object of the present invention to provide a photoacoustic imaging apparatus and a photoacoustic imaging method that enables imaging while reducing the influence of jitters of pulsed laser beams even in cases that they occur.

In order to achieve the above object, the present invention provides a photoacoustic imaging apparatus, comprising:

an ultrasound probe that includes a plurality of probe elements, which are provided to correspond to ranges of a subject to be imaged, and each of which detects acoustic signals;

a region selecting section, for sequentially selecting partial regions from among a plurality of partial regions into which a range of the subject to be imaged is divided;

a light irradiating section, for irradiating light onto a range that includes at least a selected partial region;

a light irradiation detecting section, for detecting that light has been irradiated onto the subject by the light irradiating section;

a signal obtaining section for sampling acoustic signals detected by the probe elements corresponding to the selected partial region a plurality of times over a predetermined measurement period and for storing the sampled acoustic signals in an element data memory;

an image constructing section, for reading out the plurality of pieces of data sampled with respect to each of the plurality of probe elements from the element data memory, and for constructing tomographic images of the subject based on the read out data; and

a synchronization correction processing section, for obtaining differences among the timings at which the irradiation of light is detected by the light irradiation detecting section for each of the partial regions, and for correcting the temporal axes of the pieces of sampled data within the element data memory, based on the obtained timing differences.

The synchronization correction processing section may correct the temporal axes such that the timings at which light irradiation was detected with respect to each partial region match among temporal axes of the pieces of sampled data for the partial regions in the element data memory.

Alternatively, the synchronization correction processing section may cause the signal obtaining section to shift the temporal axis for each partial region based on the timing differences and store the plurality of pieces of sampled data when the signal obtaining section stores the plurality of pieces of sampled data in the element data memory.

As a further alternative, the synchronization correction processing section may cause the image constructing section to read out the plurality of pieces of sampled data while shifting the temporal axes of the plurality of pieces of sampled data based on the timing differences when the image constructing section reads out the plurality of pieces of sampled data from the element data memory.

As a still further alternative, the synchronization correction processing section may control the sampling initiation timings of the signal obtaining section such that the amount of time between the time that light irradiation is detected by the light irradiation detecting section and the timing that sampling is initiated is the same for each of the partial regions, instead of correcting the temporal axes of the sampled data in the element data memory.

The light irradiation detecting section may be equipped with photodetectors for detecting light. Each of the photodetectors may be provided to correspond to each of the partial regions.

Alternatively, the light irradiation detecting section may be equipped with an acoustic signal detecting section for detecting acoustic signals, which are the light irradiated by the light irradiating section converted into acoustic signals at a converting section.

The number of probe elements that correspond to each partial region may be less than or equal to the number of pieces of data capable of being sampled in parallel by the signal obtaining section. The width of each partial region may be the width of a region corresponding to the number of probe elements that detect the number of pieces of data capable of being sampled in parallel by the signal obtaining section.

The present invention also provides a photoacoustic imaging method, comprising the steps of:

sequentially selecting partial regions from among a plurality of partial regions into which a range of a subject to be imaged is divided;

irradiating light onto a range that includes at least a selected partial region;

employing an ultrasound probe that includes a plurality of probe elements to detect acoustic signals from the selected partial region;

detecting the timing at which light is irradiated onto the selected partial region;

sampling the detected acoustic signals a plurality of times over a predetermined measurement period and storing the sampled acoustic signals in an element data memory;

reading out the plurality of pieces of data sampled with respect to each of the plurality of probe elements from the element data memory, and constructing tomographic images of the subject based on the read out data; and

measuring differences among the timings at which the light is irradiated onto each of the partial regions, and correcting the temporal axes of the pieces of sampled data among the partial regions within the element data memory, based on the obtained timing differences.

In the photoacoustic imaging method of the present invention, the step of correcting the temporal axes of the sampled data in the element data memory may be replaced by a step of correcting the sampling initiation timings of the signal obtaining section such that the amount of time between the time that light irradiation is detected by the light irradiation detecting section and the timing that sampling is initiated is the same for each of the partial regions.

According to the photoacoustic imaging apparatus and the photoacoustic imaging method of the present invention, the plurality of partial regions, into which the range to be imaged of a subject is divided, are sequentially selected. Irradiation of light and sampling of acoustic signals generated by the irradiation of light are performed for each selected partial region. After selection of a partial region, the light irradiation timing is detected, and differences in the light irradiation timings are measured among the partial regions. The temporal axes of the data sampled from each partial region within the element data memory are corrected based on the measured differences. By adopting this configuration, errors due to fluctuations in light irradiation timings among the partial regions can be suppressed during image construction, even if the light irradiation timings differ among the partial regions. That is, the influence of jitters can be reduced in cases that they occur, and images having high image quality can be obtained.

### BRIEF DESCRIPTION OF THE DRAWINGS

FIG. 1 is a diagram that illustrates a photoacoustic imaging apparatus according to an embodiment of the present invention.

FIG. 2 is a collection of timing charts that illustrate the relationships between pulse laser beam irradiation and data sampling for each of a plurality of partial regions.

FIG. 3 is a perspective view that illustrates an example of an ultrasound probe.

FIG. 4 is a block diagram that illustrates an example of connections between the ultrasound probe and a signal obtaining section.

FIG. 5 is a block diagram that illustrates pieces of sampled data which are stored in an element data memory.

FIG. 6 is a flow chart that illustrates the steps of the operation of the photoacoustic imaging apparatus.

### DESCRIPTION OF THE PREFERRED EMBODIMENTS

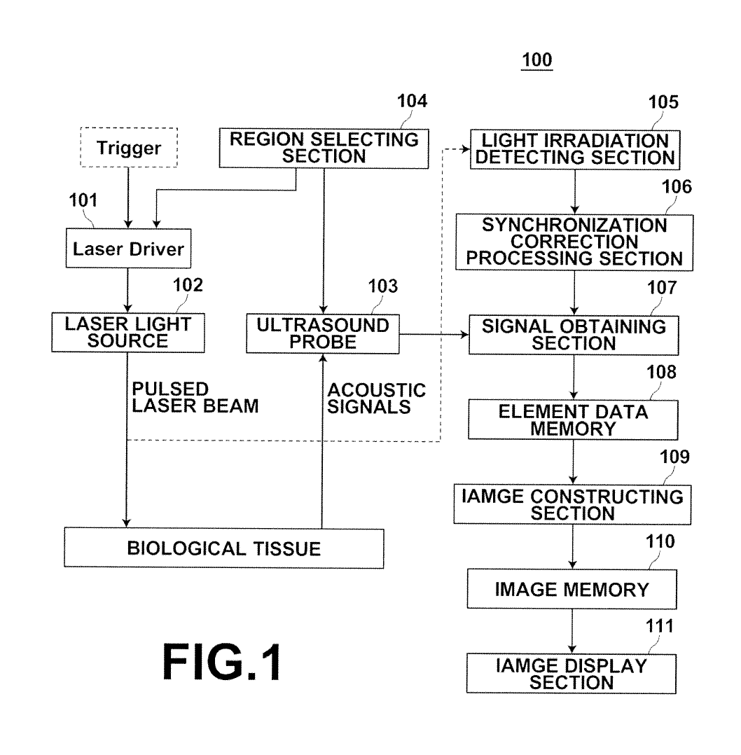

Hereinafter, embodiments of the present invention will be described in detail with reference to the attached drawings. FIG. 1 is a diagram that illustrates a photoacoustic imaging apparatus according to an embodiment of the present invention. The photoacoustic imaging apparatus 100 is equipped with: a laser driver 101; a laser light source 102; an ultrasound probe 103; a region selecting section 104; a light irradiation detecting section 105; a synchronization correction processing section 106; a signal obtaining section 107; an element data memory 108; an image constructing section 109; an image memory 110; and an image display section 111.

The laser driver 101 drives the laser light source 102. The laser light source 102 outputs a pulsed laser beam to biological tissue, which is a target of examination, when generating photoacoustic images. A Q switch solid state laser, for example, may be employed as the laser light source 102. Trigger signals are input to the laser driver 101, and the laser driver 101 drives the laser light source 102 in response to the trigger signals. The ultrasound probe 103 is equipped with ultrasound probe elements (probe elements) corresponding to a plurality of channels. The probe elements are provided corresponding to ranges of the biological tissue to be imaged. For example, the ultrasound probe 103 is equipped with 192 probe elements. The ultrasound probe 103 detects ultrasonic waves (acoustic signals) which are generated within the biological tissue by the pulsed laser beam being irradiated thereon. Each probe element converts the detected acoustic signals into electric signals, and outputs the electric signals.

The signal obtaining section 107 stores the electric signals output by the ultrasound probe 103 in the element data memory 108. The signal obtaining section 107 samples the electric signals output by the ultrasound probe a plurality of times over a predetermined measurement period, and stores the plurality of pieces of sampled data in the element data memory 108. The signal obtaining section 107 includes a preamplifier for amplifying fine signals and an A/D converter for converting analog signals into digital signals, for example. The number of signals (number of channels) capable of being sampled in parallel by the signal obtaining section 107 is less than the total number of probe elements (total number of channels) of the ultrasound probe 103. For example, in the case that the ultrasound probe 103 is equipped with 192 probe elements, the number of channels capable of being sampled in parallel by the signal obtaining section 107 is 64.

The range (the range of the biological tissue to be imaged) corresponding to the plurality of probe elements of the ultrasound probe 103 is divided into a plurality of partial regions. For example, the range to be imaged of the biological tissue is divided into three partial regions, Region A, Region B, and Region C. Region A, Region B, and Region C do not overlap each other. The width of each partial region is the width of a region corresponding to the number of probe elements that detect the number of pieces of data capable of being sampled in parallel by the signal obtaining section 107. For example, in the case that the signal obtaining section 107 is capable of sampling data for 64 channels, the width of each of the partial regions Region A, Region B, and Region C is a width corresponding to 64 probe elements.

The region selecting section 104 selects a partial region. The region selecting section 104 notifies the laser driver 101 and the ultrasound probe 103 selection data regarding a selected partial region. The laser driver 101 drives the laser light source 102 such that a pulsed laser beam is irradiated onto a range that includes at least the selected partial region. Meanwhile, the ultrasound probe 103 employs a multiplexer (not shown) or the like to connect the probe elements corresponding to the selected partial region and the signal obtaining section 107. After light is irradiated onto the partial region, the signal obtaining section 107 samples electric signals output by the probe elements connected thereto a plurality of times over a predetermined measurement period, and stores the sampled electric signals in the element data memory 108.

After the electric signals from the probe elements corresponding to the selected partial region are stored in the element data memory 108, the region selecting section 104 selects a next partial region. The region selecting section 104 sequentially selects the partial regions until the entire range to be imaged of the biological tissue is selected. Electric signals output by all of the probe elements of the ultrasound probe 103 are stored in the element data memory 108, by the region selecting section 104 sequentially selecting the partial regions. For example, the region selecting section 104 may sequentially select Region A, Region B, then Region C, and the signal obtaining section 107 may sample data for 64 channels for each region a plurality of times. Thereby, sampled data of acoustic signals corresponding to a total of 192 channels are stored in the element data memory 108.

Here, the flow of processing steps for each partial region includes: selection of a partial region; generation of a trigger signal; excitation of the laser; irradiation of a pulsed laser beam onto the biological tissue; detection of acoustic signals from the biological tissue; and storage of the electric signals in the element data memory. The timing (sampling initiation timing) at which the signal obtaining section 107 initiates obtainment of the electric signals (acoustic signals) is set in advance according to the timing at which the pulsed laser beam is irradiated onto the biological tissue. No problems will occur if the amount of time that elapses from generation of the trigger signal to the actual time when the pulsed laser beam is irradiated onto the biological tissue is constant for each partial region. However, in actuality, fluctuations occur in the laser excitation time, and there are cases in which the pulsed laser beam irradiation timings with respect to the biological tissue differ for the partial regions.

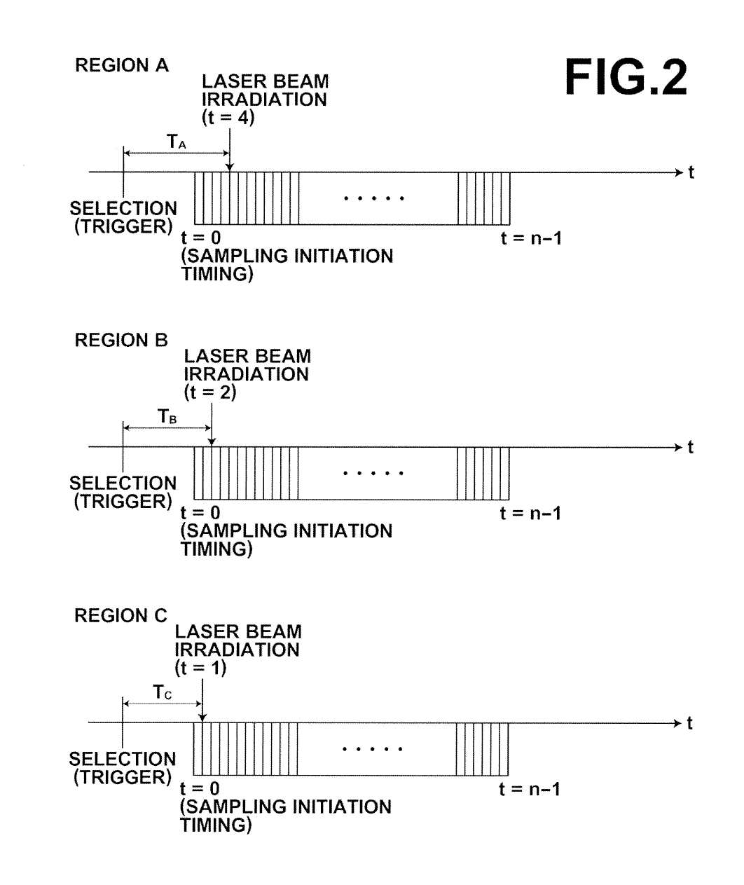

FIG. 2 is a collection of timing charts that illustrate the relationships between pulse laser beam irradiation and data sampling for each of the plurality of partial regions. Here, a case will be considered in which a range to be imaged is divided into three regions, Region A, Region B, and Region C. After the region selecting section 104 selects Region A and a trigger signal is input to the laser driver 101, a sampling initiation command is input to the signal obtaining section 107 after a predetermined amount of time elapses from the region selection timing. When the sampling initiation command is input, the signal obtaining section 107 initiates sampling of acoustic signal data. The sampling period is 50 μsec, for example. Acoustic signal data are sampled for Region B and Region C in the same manner.

The predetermined amount of time between the region selection timing and the sampling initiation timing is set based on the amount of time required for the laser light source to output the pulsed laser beam, which is estimated in advance. The sampling initiation timing for each region is defined as t=0. A first sampling operation is performed at t=0. The signal obtaining section 107 performs n sampling operations at a predetermined sampling rate during the sampling period. Thereby, the signal obtaining section 107 samples n acoustic signals between a time t=0 and t=n−1. The element data memory 108 stores n pieces of sampled data corresponding to the time between t=0 and t=n−1 for each channel.

Ideally, the timing at which the pulsed laser beam is irradiated onto each partial region after it is selected is constant. However, if jitters occur in the pulsed laser beam, the amount of time that elapses from selection of a partial region and actual irradiation of the pulsed laser beam will not always be the same. In such cases, the relationship between the sampling initiation timing and the light irradiation timing will become shifted among the partial regions. For example, the laser irradiation timing may be t=4 in Region A, whereas the laser irradiation timing may be t=2 in Region B, as illustrated in FIG. 2. In photoacoustic imaging, acoustic waves are generated by biological tissue by the biological tissue absorbing pulsed laser beams. Therefore, errors will be generated if the light irradiation timings are shifted among partial regions. The present embodiment corrects these shifts using the light irradiation detecting section 105 and the synchronization correction processing section 106 illustrated in FIG. 1.

The light irradiation detecting section 105 detects irradiation of the pulsed laser beam from the laser light source 102 onto the biological tissue. The light irradiation detecting section 105 is provided in the vicinity of the portion of the biological tissue onto which the pulsed laser beam from the laser light source 102 is irradiated. The light irradiation detecting section 105 may be provided corresponding to each of the partial regions. For example, in the case that the range to be imaged is divided into the three partial regions Region A, Region B, and Region C, the light irradiation detection section 105 may be provided corresponding to each the three partial regions. A photodetector that outputs an electric signal when light is detected may be employed as the light irradiation detecting section 105.

The synchronization correction processing section 106 obtains the timing at which the light irradiation detecting section 105 has detected irradiation of light for each of the partial regions, and derives the differences in the light irradiation detection timings among the partial regions. Here, the light irradiation timing of a partial region may be defined as the amount of time between a synchronization point and the time at which light irradiation is detected by the light irradiation detecting section 105 for each partial region. In other words, the light irradiation timing for a partial region can be defined as the time at which light irradiation is detected by the light irradiation detecting section 105 when the synchronization point is defined as time 0. The differences in the light irradiation timings among the partial regions may be defined as the difference in the time at which light irradiation is detected by the light irradiation detecting section 105 for a partial region and the time at which light irradiation is detected by the light irradiation detecting section 105 for another partial region, when the synchronization point is defined as 0.

For example, the synchronization correction processing section 106 calculates the amount of time between a time which is a synchronization point (reference) and a time at which the light irradiation detecting section 105 detects that the pulse laser beam has been irradiated onto the biological tissue, for each partial region. The time to be the reference may be the timings when the trigger signals are input to the laser driver 101 after the partial regions are selected, for example. Alternatively, the reference time may be the timings when signals that indicate initiation of signal obtainment (initiation of sampling) are input to the signal obtaining section 107. The synchronization correction processing section 106 obtains the amount of time from the reference time until the pulsed laser beam is irradiated onto the biological tissue for each partial region, and obtains the differences in the amounts of time among the partial regions as the differences in the light irradiation timings.

The synchronization correction processing section 106 corrects the temporal axes of the acoustic signal data sampled by the signal obtaining section 107 among the partial regions, based on the light irradiation timing differences among the partial regions. More specifically, the synchronization correction processing section 106 corrects the temporal axes for each partial region based on the differences in the detected light irradiation timings of the pulsed laser beam, when storing the plurality of pieces of acoustic signal data sampled by the signal obtaining section 107 in the element data memory 108. The synchronization correction processing section 106 corrects the temporal axes of the sampled pieces of acoustic signal data to be stored in the element data memory 108 such that the timings at which the pulsed laser beam is irradiated onto the biological tissue match among the partial regions.

The image constructing section 109 initiates image construction after the region selecting section 104 has selected all of the partial regions, and the signal obtaining section 107 has sampled the acoustic signals detected by the probe elements of the range of the biological tissue to be imaged and has stored the sampled acoustic signals in the element data memory 108. The image constructing section 109 reads out the plurality of pieces of sampled data obtained from probe elements corresponding to 192 channels, for example, from the element data memory 109, and generates a tomographic image of the biological tissue based on the read out data. The image constructing section 109 typically includes a signal processing section, a phase matching adding section, and an image processing section. A description of the detailed procedures involved in image construction performed by the image constructing section 109 will be omitted. The functions of the image constructing section 109 can be realized by a computer operating according to a predetermined program. Alternatively, the functions of the image constructing section 109 may be realized by a DSP (Digital Signal Processor), an FPGA (Field Programmable Gate Array), or the like. The image constructing section 109 stores the generated photoacoustic image in the image memory 110. The image display section 111 displays the tomographic image stored in the image memory 110 on a display monitor or the like.

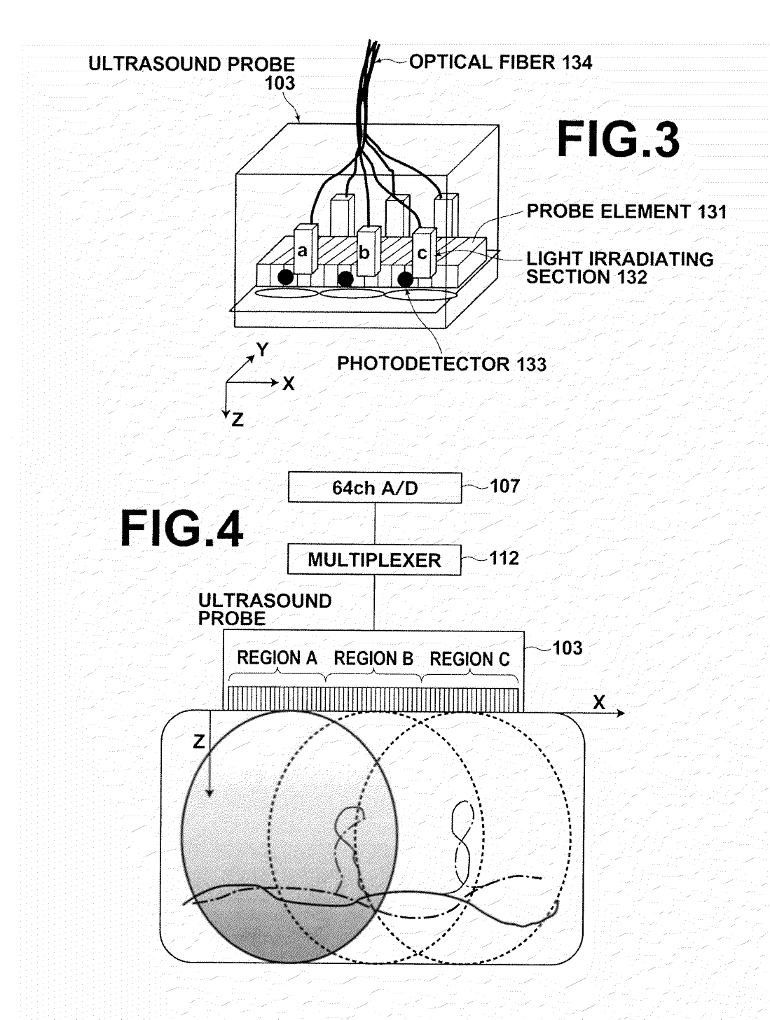

FIG. 3 illustrates the ultrasound probe 103. The ultrasound probe 103 is equipped with the plurality of probe elements 131. The probe elements 131 are arranged unidirectionally along a predetermined direction, for example. Optical fibers 134 guide light output by the laser light source 102 (refer to FIG. 1) to light irradiating sections 132 provided within the ultrasound probe 103. The light irradiating sections 132 irradiate the pulsed laser beam output by the laser light source 102 onto regions that at least include a selected partial region. The light irradiating sections 132 are provided corresponding to each of Region A, Region B, and Region C, for example. In this case, the light irradiating section 132 corresponding to Region A irradiates the pulse laser beam onto at least Region A when Region A is selected. The light irradiating section 132 corresponding to Region B irradiates the pulse laser beam onto at least Region B when Region B is selected, and the light irradiating section 132 corresponding to Region C irradiates the pulse laser beam onto at least Region C when Region C is selected.

Photodetectors 133 are included in the light irradiation detecting section 105 illustrated in FIG. 1. The photodetectors 133 detect that light is irradiated onto biological tissue from the light irradiating sections 132. The photodetectors 133 output signals indicated that light is detected when they receive light from the light irradiating sections 132, for example. The photodetectors 133 may be provided corresponding to each of the partial regions. For example, in the case that the range to be imaged is divided into the three partial regions Region A, Region B, and Region C, a photodetector 133 may be provided corresponding to each the three partial regions. The photodetector 133 corresponding to Region A detects irradiation of the pulsed laser beam from the light source 102 by the light irradiating section 132 onto Region A when Region A is selected. The photodetectors 133 corresponding to Region B and Region C respectively detect that the pulsed laser beam is irradiated onto the regions that they correspond to, when the regions are selected.

FIG. 4 is a block diagram that illustrates an example of connections between the ultrasound probe 103 and the signal obtaining section 107. The ultrasound probe 103 is equipped with probe elements 131 (refer to FIG. 2) for 192 channels, for example. The width corresponding to the probe elements 131 is divided into three partial regions (Regions A through C), and the width of each partial region is a width that corresponds to probe elements 131 for 64 channels. If the width of the biological tissue corresponding to the probe elements 131 for 192 channels is 57.6 mm, the width of each partial region will be 19.2. The biological data imaging apparatus 100 performs irradiation of light onto and data collection from the 19.2 mm wide partial regions divided as illustrated in FIG. 4 three times, to obtain data for all 192 channels.

The signal obtaining section 107 includes an A/D converter capable of sampling data for 64 channels in parallel. A multiplexer 112 selectively connects the probe elements of the ultrasound probe 103 and the signal obtaining section 107. The multiplexer 112 is connected to the probe elements corresponding to 192 channels, for example, and selectively connects 64 channels to the A/D converter of the signal obtaining section 107. For example, when Region A is selected, the multiplexer 112 connects the probe elements of the 64 channels corresponding to Region A to the AD converter of the signal obtaining section 107. When Region B is selected, the multiplexer 112 connects the probe elements of the 64 channels corresponding to Region B to the AD converter of the signal obtaining section 107, and when Region C is selected, the multiplexer 112 connects the probe elements of the 64 channels corresponding to Region C to the AD converter of the signal obtaining section 107.

If Region A is selected, and the light irradiating section 132 (refer to FIG. 3) irradiates a pulsed laser beam onto Region A of the biological tissue, the laser beam propagates with a certain degree of spread due to scattering within the biological tissue. Absorbers such as blood that exist within the biological tissue absorb the energy of the pulsed laser beam, and generate acoustic signals. The amount of time required before these acoustic signals are detected by the probe elements is determined according to the positional relationship between the acoustic signal generation point and the probe elements in the X direction, and the position of the acoustic signal generating point in the Z direction. Electric signals output by the probe elements 131 selected by the multiplexer 112 are sampled a plurality of times over a predetermined measurement period, in order to detect these acoustic signals. Acoustic signals are detected for Region B and Region C in a similar manner, by irradiating a pulsed laser beam onto these regions, and by sampling electric signals output by probe elements corresponding to each of the regions over a predetermined measurement period.

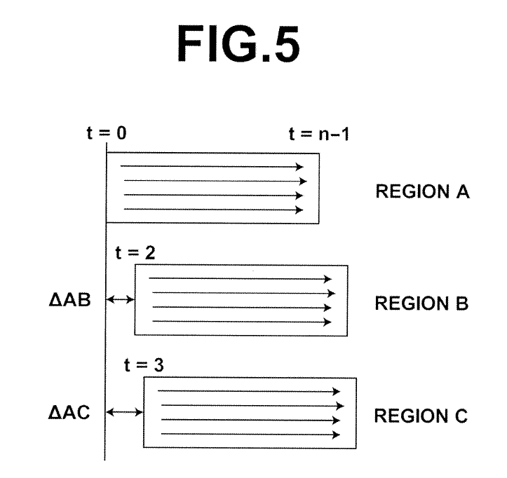

FIG. 5 is a block diagram that illustrates pieces of data which are stored in the element data memory 108. The element data memory 108 has stored therein n pieces of sampled data corresponding to timings t=0 through t=n−1, obtained from each probe element of the ultrasound probe 103. Here, a case will be described in which that the region selecting section 104 sequentially selects Region A, Region B, and Region C. In this case, the signal obtaining section 107 first obtains n pieces of sampled data for the probe elements (for example, probe elements corresponding to 64 channels) for Region A. At this time, the synchronization correction processing section 106 obtains the amount of time (T_{A}) that elapses between the time that Region A was selected and the time at which light is actually irradiated onto Region A. The signal obtaining section 107 stores n pieces of sampled data at locations (addresses) within the element data memory 108 corresponding to each timing between t=0 and t=n−1.

Next, when Region B is selected, the signal obtaining section 107 obtains n pieces of sampled data for the probe elements (for example, probe elements corresponding to 64 channels) for Region B. At this time, the synchronization correction processing section 106 obtains the amount of time (T_{B}) that elapses between the time that Region A was selected and the time at which light is actually irradiated onto Region B. This amount of time T_{B} corresponds to the relationship between the sampling initiation timing for Region B and the timing at which light is actually irradiated onto Region B. The synchronization correction processing section 106 obtains the amount that the light irradiation timing of Region B is shifted from the light irradiation timing of Region A, using the light irradiation timing of Region A as a reference.

The synchronization correction processing section 106 obtains the difference between the amount of time T_{A} obtained with respect to Region A and the amount of time T_{B} obtained with respect to Region B as the difference in light irradiation timings between Region A and Region B. For example, assume that the light irradiation detecting section 105 detects light irradiation at a time (t=4) one sampling cycle from initiation of sampling in Region A, as illustrated in FIG. 2. In addition, assume that the light irradiation detecting section 105 detects light irradiation at a time (t=2) two sampling cycles from initiation of sampling in Region B. In such a case, the difference (ΔAB) in light irradiation timings between Region A and Region B is calculated as ΔAB=T_{B}−T_{A}=−2.

The synchronization correction processing section 106 shifts the temporal axis of the sampled data of Region B within the element data memory 108 from the temporal axis of the sampled data of Region A for an amount corresponding to the calculated difference in light irradiation timings, and causes the signal obtaining section 107 to store the obtained sampled data. In the case described above, the synchronization correction processing section 106 delays the temporal axis in the element data memory 108 for two sampling cycles when the sampled data of Region B are stored. Thereby, the signal obtaining section 107 stores the sampled data of Region B from t=2 in the element data memory 108, as illustrated in FIG. 5. Note that timings for which data are not stored may be filled with blank signals (having data values of 0). In addition, data which cannot be stored in the element data memory 108 due to the delay in the data storage timing, for example, data for t=n−2 and thereafter of Region B, may be discarded.

When Region C is selected after Region B, the signal obtaining section 107 obtains n pieces of sampled data for the probe elements (for example, probe elements corresponding to 64 channels) for Region C. The synchronization correction processing section 106 obtains the difference in light irradiation timings between Region A and Region C using the light irradiation timing of Region A as a reference, in the same manner as for Region B. For example, assume that the light irradiation detecting section 105 detects light irradiation at a time (t=1) one sampling cycle from initiation of sampling in Region C, as illustrated in FIG. 2. In such a case, the difference (ΔAC) in light irradiation timings between Region A and Region C is calculated as ΔAC=T_{C}−T_{A}=−3. In this case, the synchronization correction processing section 106 delays the temporal axis in the element data memory 108 for one sampling cycle when the sampled data of Region C are stored. Thereby, the signal obtaining section 107 stores the sampled data of Region C from t=3 in the element data memory 108, as illustrated in FIG. 5.

The synchronization correction processing section 106 corrects the temporal axes in the element data memory 108 as described above. Thereby, the fourth piece of sampled data of Region A, the second piece of sampled data of Region B, and the first piece of sampled data of Region C are stored in the element data memory 108 as data for a timing t=4. The synchronization correction processing section 106 corrects the temporal axis in the element data memory 108 for each partial region when storing the data. Thereby, the timings at which the pulsed laser beam was irradiated onto the biological tissue in each partial region can be caused to match among the partial regions along the temporal axes of the sampled data within the element data memory 108.

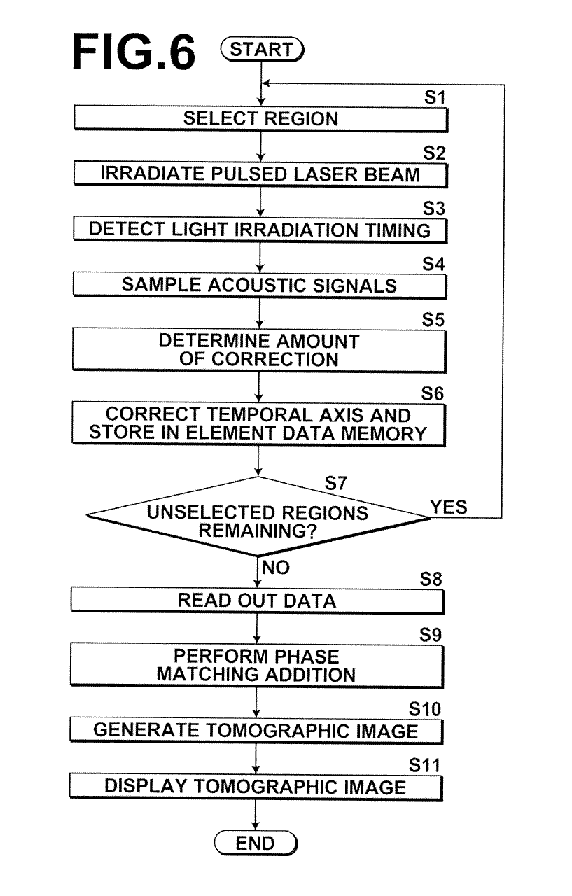

FIG. 6 is a flow chart that illustrates the steps of the operation of the photoacoustic imaging apparatus 100. The steps can be broadly divided into a data collecting phase, and an image constructing phase that performs image construction based on collected data. The region selecting section 104 selects one of the partial regions (step S1). The laser driver 101 drives the laser light source 102, and the laser light source 102 irradiates a pulsed laser beam onto a range that at least includes the partial region selected in step S1 (step S2). The synchronization correction processing section 106 employs the light irradiation detecting section 105 to detect the timing at which the pulsed laser beam is irradiated onto the biological tissue (step S3). Acoustic signals are generated within the biological tissue by the pulsed laser bema being irradiated thereon. The acoustic signals are detected by the probe elements of the ultrasound probe 103. The signal obtaining section 107 samples the signals output by the probe elements a plurality of times over a predetermined measurement period (step S4). Steps S3 and S4 may be performed in parallel.

In the case that a previously selected partial region exists, the synchronization correction processing section 106 obtains the difference in the light irradiation timings with respect to the previously selected partial region, and determines an amount of correction for the temporal axis of the sampled data within the element data memory (step S5). In the case that the partial region which was selected in step S1 is the first partial region, the light irradiation timing of the partial region is employed as a reference, and the amount of correction may be determined as 0 (no correction). The signal obtaining section 107 stores the plurality of pieces of sampled data sampled in step S4 in the element data memory 108 (step S6). At this time, the signal obtaining section 107 corrects the temporal axis in the element data memory 108 according to the amount of correction determined in step S5.

The region selecting section 104 judges whether there are any partial regions which have not been selected yet (step S7). In the case that a partial region which has not been selected yet exists, the process returns to step S1, and the region selecting section 104 selects one of the as of yet unselected partial regions. Thereafter, steps S2 through S4 are executed, and then the synchronization correction processing section 106 obtains the difference between the reference light irradiation timing and the light irradiation timing of the presently selected partial region and determines the amount of correction for the temporal axis of the sampled data in the element data memory at step S5. The signal obtaining section 107 shifts the temporal axis of the sampled data in the element data memory 108 for the determined amount of correction, and stores the plurality of pieces of sampled data in the element data memory 108.

The photoacoustic imaging apparatus 100 repeatedly executes steps S1 through S6 until there are no more remaining unselected partial regions, to store sampled data of each partial region in the element data memory 108. When it is judged that there are no more partial regions which have not been selected yet at step S7, that is, when it is judged that all of the partial regions have been selected, the region selecting section 104 transfers processing to the image constructing section 109. The steps up to this point correspond to the data collecting phase. The plurality of pieces of sampled data from the probe elements of the ultrasound probe 103 corresponding to 192 channels are stored in the element data memory 108 by the steps up to this point.

When data collection is complete, the image constructing section 109 reads out the sampled data from the element data memory 108, and initiates image construction (step S8). The image constructing section 109 performs phase matching addition employing sampled data for a predetermined number of channels (step S9), and generates a tomographic image (step S10). The image constructing section 109 stores the generated tomographic image in the image memory 110. As necessary, the image display section 111 reads out the tomographic image from the image memory 110 and displays the tomographic image on a display or the like (step S11). Steps S8 through S11 correspond to the image construction phase.

In the present embodiment, the range to be imaged of the biological tissue is divided into the plurality of partial regions. The region selecting section 104 sequentially selects the partial regions, irradiation of light and detection of acoustic signals are performed for each partial region, and the sampled acoustic signals are stored in the element data memory 108 for each partial region. The synchronization correction processing section 106 employs the light irradiation detecting section 105 to obtain the differences among the timings at which light irradiation was detected among the partial regions. Then, the temporal axes of the sampled data in the element data memory 108 are corrected based on the obtained differences in the light irradiation timings. The timings at which light irradiation was detected in each of the partial regions can be caused to match along the temporal axes within the element data memory, by the corrections being performed. For this region, even in cases that jitters occur in the pulsed laser beam which is irradiated onto each partial region, the influence thereof can be reduced when imaging is performed.

In the present embodiment, the range to be imaged of the biological tissue is divided into the plurality of partial regions. Therefore, it is sufficient for the number of signals which are sampled in parallel by the signal obtaining section 107 to be that of the number of probe elements corresponding to each of the partial regions. For example, if the ultrasound probe 103 has probe elements corresponding to 192 channels and the width of each partial region corresponds to probe elements corresponding to 64 channels, the signal obtaining section 107 need only be capable of sampling data for 64 channels in parallel . Circuits for obtaining a great number of pieces of data in parallel and at high speed are expensive. In the present embodiment, the number of pieces of data which is sampled in parallel by the signal obtaining section 107 can be less than the total number of probe elements of the ultrasound probe 103. Therefore, costs can be reduced compared to a case in which the signal obtaining section 107 is configured to obtain a number of signals corresponding to all of the probe elements of the ultrasound probe 103 in parallel.

In the present embodiment, data necessary to construct one image can be sampled and stored in the element data memory 108 by irradiating the pulsed laser beam a number of times equal to the number of partial regions at minimum. For this reason, the amount of time required to obtain an image can be reduced compared to a case in which the range of data collection is shifted one line at a time, phase matching is performed, and an image is constructed, as in the method disclosed in Japanese Unexamined Patent Publication No. 2005-21380. The present embodiment enables sufficiently high speed imaging, if laser repetition of 1 kHz, which is a condition of safety, can be realized. Because the speed of obtaining an image is fast in the present embodiment, influence of movements (motion artifacts) can be suppressed, and images of subjects that exhibit movement can be favorably obtained.

In the present embodiment, when a certain partial region is selected, it is only necessary to irradiate a pulsed laser beam onto at least the selected partial region. That is, it is not necessary to irradiate the entire range of the biological tissue with the laser beam. For example, a pulsed laser beam on the order of nanoseconds is necessary for photoacoustic imaging. A Q switch solid state laser is an example of a light source to be employed to irradiate such a pulsed laser beam. In the case that the pulsed laser beam is to be irradiated onto the entire range of the biological tissue and power of 20 mj/cm², which is the safety standard of the Q switch solid state laser, is to be obtained, pulsed output of 60 mJ or greater will be necessary, considering the efficiency of optical systems and the irradiation range. This will become a factor in increasing the cost of the apparatus. In the present embodiment, it is possible to irradiate the pulsed laser beam onto each partial region by switching the irradiation range, thereby suppressing the power of the light source. This is advantageous from the viewpoint of cost.

In the present embodiment, the amount of time required to construct an image is short, and it is possible to obtain images at speeds faster than that practically necessary, as determined depending on the targets of diagnosis. In the case that it is possible to perform imaging at speeds greater than a practically necessary imaging speed, steps S2 through S6 illustrated in FIG. 6 may be performed a plurality of times for each of Region A, Region B, and Region C, for example, and averages of the plurality of pieces of sampled data may be obtained and stored in the element data memory 108. Alternatively, steps S1 through S10 of FIG. 6 maybe repeatedly executed a plurality of times to generate a plurality of tomographic images, and the plurality of generated tomographic images may be averaged. In the case that such a configuration is adopted, the S/N ratio (Signal to Noise ratio) of the image may be improved, enabling obtainment of high image quality to become possible.

In the embodiment described above, the partial regions are set such that they do not overlap each other. However, the present invention is not limited to such a configuration. The partial regions may include regions that overlap with other partial regions. For example, if the ultrasound probe 103 has probe elements corresponding to 192 channels, the range to be imaged may be divided into five partial regions. The first through 64th probe elements may be designated as Region A, the 32nd through 96th probe elements may be designated as Region B, the 96th through 128th probe elements may be designated as Region C, the 128th through 160th probe elements may be designated as Region D, and the 160th through 192nd probe elements may be designated as Region E. In this case, for example, the 32nd through 64th probe elements overlap between Region A and Region B, and the 64th and 96th probe elements overlap between Region B and Region C. In the case that the regions overlap as described above, because the 32nd through 64th probe elements overlap between Region A and Region B, for example, data sampled when the pulsed laser beam is irradiated onto Region A and data sampled when the pulsed laser beam is irradiated onto Region B are obtained from the probe elements in the overlapping region. The data of the overlapping regions can enable improvements in SIN ratio, by averaging the plurality of pieces of sampled data, for example. However, as the overlaps among the partial regions increase, the number of pulsed laser beam irradiations and data sampling operations will increase. Therefore, the imaging speed will deteriorate. Whether the partial regions are to have overlapping regions, or the degree of overlap among the partial regions may be set as appropriate according to desired imaging speed and the like.

In addition, in the case described above, if the sampled data from the 40th probe element is considered, for example, this probe element should detect acoustic signals at the same timing when the pulsed laser light is irradiated onto Region A and when the pulsed laser light is irradiated onto Region B. Accordingly, it is possible to estimate the light irradiation timings among the partial regions, by obtaining the differences in the detection timings of the photoacoustic signals. When obtaining the differences among the light irradiation timings of the partial regions, the acoustic signal detection timings in the overlapping regions may be utilized in addition to the light irradiation timings detected by the light irradiation detecting section 105.

In the embodiment described above, the synchronization correction processing section 106 shifts the temporal axis of each partial region based on the differences in the light irradiation timings among the partial regions. However, the present invention is not limited to such a configuration. For example, the plurality of pieces of sampled date may be stored in the element data memory 108 without the temporal axes thereof being corrected, and the temporal axes of the sampled data may be corrected when the image constructing section 109 reads out the data from the element data memory 108. That is, the synchronization correction processing section 106 may cause the image constructing section 109 to shift the temporal axis of each partial region based on the light irradiation timings of each partial region when reading out the plurality of pieces of sampled data from the element data memory 108.

For example, assume a case in which the light irradiation timings are t=4 for Region A, t=2 for Region B, and t=1 for Region C, as illustrated in FIG. 2. The element data memory 108 stores the n pieces of sampled data for each region from t=0. In the case that Region A is employed as a reference, the synchronization correction processing section 106 determines the amount of correction to be 0 (no correction) when reading out data from the probe elements corresponding to Region A. In this case, data of t=0 stored in the element data memory 108 are read out as is, that is, data of t=0. The synchronization correction processing section 106 sets the amount of correction to be 2 when reading out data from the probe elements corresponding to Region B. In this case, data of t=0 stored in the element data memory 108 are read out as data of t=2. The synchronization correction processing section 106 sets the amount of correction to be 3 when reading out data from the probe elements corresponding to Region C. In this case, data of t=0 stored in the element data memory 108 are read out as data of t=3.

By correcting the temporal axis during readout as described above, the image constructing section 109 reads out data obtained during a fourth sampling cycle in Region A, data obtained during a second sampling cycle in Region B, and data obtained during a first sampling cycle in Region C as data of time t=4. In the case that the synchronization correction processing section 106 corrects the temporal axes in the element data memory 108 during readout as well, image construction in a state in which the timings at which the pulsed laser beam was irradiated onto the biological tissue are matched among the partial regions is enabled, in the same manner as the case in which the temporal axes are corrected during data storage.

In the embodiment described above, the timings at which the signal obtaining section 107 initiates sampling were set to be constant, independent of the actual light irradiation timings. Instead, the synchronization correction processing section 106 may control the sampling initiation timings such that the amount of time between the time that light irradiation is detected by the light irradiation detecting section 105 and the timing that sampling is initiated by the signal obtaining section 107 is the same for each of the partial regions. In the case that control is exerted in this manner, the timings at which the pulsed laser beam was irradiated onto the biological tissue in each partial region along the temporal axes of the sampled data in the element data memory 108 can be caused to match even if the synchronization correction processing section 106 does not correct the temporal axes of the sampled data in the element data memory 108 among the partial regions.

The above embodiment was described as an example in which the light irradiation detecting section 105 included photodetectors. However, the present invention is not limited to this configuration. The light irradiation detecting section 105 needs only to detect that light has been irradiated onto the biological tissue, and direct detection of light is not necessary. For example, the light irradiation detecting sectionmay be equipped with an acoustic signal detecting section, and the acoustic signal detecting section may detect acoustic signals, which are the light irradiated by the light irradiating section 132 (refer to FIG. 3) converted into acoustic signals at a converting section. The converting section for converting light into acoustic signals may be provided in the interior of the ultrasound probe 103, for example. Alternatively, the converting section for converting light into acoustic signals may be adhesively attached to the surface of the biological tissue. That is, acoustic signals, which have been converted into acoustic signals by the converting section that convert light into acoustic signals, that indicate that light have been irradiated may be detected by the probe elements 131.

The present invention has been described based on a preferred embodiment thereof. However, the photoacoustic imaging apparatus and the photoacoustic imaging method of the present invention are not limited to the above embodiments. Various modifications and changes may be added to the configurations of the above embodiments, as long as they do not stray from the spirit and scope of the inventions as claimed below.

What is claimed is:

1. A photoacoustic imaging apparatus, comprising:
an ultrasound probe that includes a plurality of probe elements, which are provided to correspond to ranges of a subject to be imaged, and each of which detects acoustic signals;
a region selecting section, for sequentially selecting partial regions from among a plurality of partial regions into which a range of the biological tissue to be imaged is divided;
a light irradiating section, for irradiating light onto a range that includes at least a selected partial region;
a light irradiation detecting section, for detecting that light has been irradiated onto the subject by the light irradiating section;
a signal obtaining section for sampling acoustic signals detected by the probe elements corresponding to the selected partial region a plurality of times over a predetermined measurement period and for storing the sampled acoustic signals in an element data memory;
an image constructing section, for reading out the plurality of pieces of data sampled with respect to each of the plurality of probe elements from the element data memory, and for constructing tomographic images of the subject based on the read out data; and
a synchronization correction processing section, for obtaining differences among the timings at which the irradiation of light is detected by the light irradiation detecting section for each of the partial regions, and for correcting the temporal axes of the pieces of sampled data within the element data memory, based on the obtained timing differences.

2. A photoacoustic imaging apparatus as defined in claim 1, wherein:
the synchronization correction processing section corrects the temporal axes such that the timings at which light irradiation was detected with respect to each partial region match among temporal axes of the pieces of sampled data for the partial regions in the element data memory.

3. A photoacoustic imaging apparatus as defined in claim 1, wherein:
the synchronization correction processing section causes the signal obtaining section to shift the temporal axis for each partial region based on the timing differences and stores the plurality of pieces of sampled data when the signal obtaining section stores the plurality of pieces of sampled data in the element data memory.

4. A photoacoustic imaging apparatus as defined in claim 1, wherein:
the synchronization correction processing section causes the image constructing section to read out the plurality of pieces of sampled data while shifting the temporal axes of the plurality of pieces of sampled data based on the timing differences when the image constructing section reads out the plurality of pieces of sampled data from the element data memory.

5. A photoacoustic imaging apparatus as defined in claim 1, wherein the synchronization correction processing section controls the sampling initiation timings of the signal obtaining section such that the amount of time between the time that light irradiation is detected by the light irradiation detecting section and the timing that sampling is initiated is the same for each of the partial regions, instead of correcting the temporal axes of the sampled data in the element data memory.

6. A photoacoustic imaging apparatus as defined in claim 1, wherein:
the light irradiation detecting section is equipped with photodetectors for detecting light.

7. A photoacoustic imaging apparatus as defined in claim 6, wherein:
each of the photodetectors is provided to correspond to each of the partial regions.

8. A photoacoustic imaging apparatus as defined in claim 1, wherein:
the light irradiation detecting section is equipped with an acoustic signal detecting section for detecting acoustic signals, which are the light irradiated by the light irradiating section converted into acoustic signals at a converting section.

9. A photoacoustic imaging apparatus as defined in claim 1, wherein:
the number of probe elements that correspond to each partial region is less than or equal to the number of pieces of data capable of being sampled in parallel by the signal obtaining section.

10. A photoacoustic imaging apparatus as defined in claim 1, wherein:
the width of each partial region is the width of a region corresponding to the number of probe elements that detect the number of pieces of data capable of being sampled in parallel by the signal obtaining section.

11. A photoacoustic imaging apparatus as defined in claim 1, wherein:
the plurality of partial regions do not overlap each other.

12. A photoacoustic imaging apparatus as defined in claim 1, wherein:
the plurality of partial regions have overlapping portions.

13. A photoacoustic imaging apparatus as defined in claim 1, wherein:
the signal obtaining section is equipped with an A/D converter; and
the probe elements corresponding to the selected partial region and the A/D converter are selectively connected to each other by a multiplexer.

14. A photoacoustic imaging apparatus as defined in claim 1, wherein:
the number of probe elements corresponding to at least one of the partial regions is different from the number of probe elements corresponding to another one of the partial regions.

15. A photoacoustic imaging apparatus as defined in claim 1, wherein:
the pieces of sampled data are averaged.

16. A photoacoustic imaging apparatus as defined in claim 1, wherein:
the light irradiating section is equipped with a Q switch solid state laser.

17. A photoacoustic imaging apparatus as defined in claim 1, wherein:
a reference time for the timings is a timing at which trigger signals are input to the light irradiating section.

18. A photoacoustic imaging apparatus as defined in claim 1, wherein:
a reference time for the timings is a timing at which signals that indicate initiation of signal obtainment are input to the signal obtaining section

19. A photoacoustic imaging method, comprising the steps of:
sequentially selecting partial regions from among a plurality of partial regions into which a range of a subject to be imaged is divided;
irradiating light onto a range that includes at least a selected partial region;
employing an ultrasound probe that includes a plurality of probe elements to detect acoustic signals from the selected partial region;
detecting the timing at which light is irradiated onto the selected partial region;
sampling the detected acoustic signals a plurality of times over a predetermined measurement period and storing the sampled acoustic signals in an element data memory;
reading out the plurality of pieces of data sampled with respect to each of the plurality of probe elements from the element data memory, and constructing tomographic images of the subject based on the read out data; and
measuring differences among the timings at which the light is irradiated onto each of the partial regions, and correcting the temporal axes of the pieces of sampled data among the partial regions within the element data memory, based on the obtained timing differences.

20. A photoacoustic imaging method as defined in claim 18, wherein:
the step of correcting the temporal axes of the sampled data in the element data memory is replaced by a step of correcting the sampling initiation timings of the signal obtaining section such that the amount of time between the time that light irradiation is detected by the light irradiation detecting section and the timing that sampling is initiated is the same for each of the partial regions.
